(19) United States
(12) Patent Application Publication
(10) Pub. No.: US 2023/0397906 A1
(43) Pub. Date: Dec. 14, 2023

(54) ROBOT PRESSING MECHANISM, NASOPHARYNGEAL SWAB SAMPLING APPARATUS INCLUDING THE SAME, AND NASOPHARYNGEAL SWAB SAMPLING METHOD USING THE SAME

(71) Applicant: KOREA INSTITUTE OF SCIENCE AND TECHNOLOGY, Seoul (KR)

(72) Inventors: Jongwon LEE, Seoul (KR); Yong-Jae KIM, Cheonan-si (KR); Chan-Young MAENG, Cheonan-si (KR); JongJun YOON, Hanam-si (KR)

(21) Appl. No.: 18/304,199

(22) Filed: Apr. 20, 2023

(30) Foreign Application Priority Data
Jun. 10, 2022 (KR) 10-2022-0070557

(51) Int. Cl. A61B 10/00; A61F 13/38

(52) U.S. Cl. CPC A61B 10/0051; A61F 13/38; A61B 2034/302

(57) ABSTRACT

Provided is a robot pressing mechanism which includes a pressing device extending in a first direction, a support device connected to the pressing device and moving in the first direction relative to the pressing device, and a flat spring configured to connect the pressing device to the support device. The support device includes a guide member spaced apart from the pressing device in a direction crossing the first direction. The flat spring includes a first plate coupled to the pressing device and extending in the first direction, a second plate coupled to the guide member and extending in a direction in which the guide member extends, and a third plate bent to connect one end of the first plate to one end of the second plate.

### CROSS-REFERENCE TO RELATED APPLICATIONS

This U.S. non-provisional patent application claims priority under 35 U.S.C. § 119 of Korean Patent Application No. 10-2022-0070557, filed on Jun. 10, 2022, the entire contents of which are hereby incorporated by reference.

### BACKGROUND

The present disclosure herein relates to a robot pressing mechanism, a nasopharyngeal swab sampling apparatus including the same, and a nasopharyngeal swab sampling method using the same and, more particularly, to a robot pressing mechanism capable of exerting constant force, a nasopharyngeal swab sampling apparatus including the same, and a nasopharyngeal swab sampling method using the same.

It may be necessary to extract a sample from the human body to confirm whether the human body is infected with a coronavirus or the like. For example, the sample may be extracted from the mucous membrane in the nasal cavity of the human body. From the extracted sample, it is possible to determine whether the human body is infected with various viruses. A swab may be used to extract a sample from the mucous membrane in the nasal cavity of the human body. More specifically, when the swab is rotated after being inserted into the nasal cavity, the sample may be smeared on one end of the swab. This process is manually performed by a medical staff, and thus it may take a lot of time.

### SUMMARY

The present disclosure provides a robot pressing mechanism capable of pressing a target at uniform force, a nasopharyngeal swab sampling apparatus including the same, and a nasopharyngeal swab sampling method using the same.

The present disclosure also provides a robot pressing mechanism capable of preventing damage to the mucous membrane of the human body, a nasopharyngeal swab sampling apparatus including the same, and a nasopharyngeal swab sampling method using the same.

The present disclosure also provides a robot pressing mechanism capable of preparing for unexpected movements of the human body, a nasopharyngeal swab sampling apparatus including the same, and a nasopharyngeal swab sampling method using the same.

The objects of the present disclosure are not limited to the aforementioned objects, but other objects not described herein will be clearly understood by those skilled in the art from the following description.

An embodiment of the inventive concept provides a robot pressing mechanism including: a pressing device extending in a first direction; a support device connected to the pressing device and moving in the first direction relative to the pressing device; and a flat spring configured to connect the pressing device to the support device, wherein the support device includes a guide member that is spaced apart from the pressing device in a direction crossing the first direction, wherein the flat spring includes: a first plate coupled to the pressing device and extending in the first direction; a second plate coupled to the guide member and extending in a direction in which the guide member extends; and a third plate bent to connect one end of the first plate to one end of the second plate.

In an embodiment, the width of the third plate may be not constant.

In an embodiment, the width of the third plate may increase in a direction from the first plate to the second plate.

In an embodiment, the third plate may have a trapezoidal shape so that the width of the third plate increases constantly in the direction from the first plate to the second plate.

In an embodiment, the guide member may have a plate shape, wherein the guide member extends in the first direction, and the inner surface of the guide member is parallel to the pressing device.

In an embodiment, the first plate may be coupled to the outer surface of the pressing device, and the second plate may be coupled to the inner surface of the guide member, wherein the inner surface of the guide member is not parallel to the outer surface of the pressing device, and the first plate is not parallel to the second plate.

In an embodiment, the support device may further include a support body configured to support the guide member, wherein the pressing device passes through the support body in the first direction, and the guide member is connected to the support body and rotates relative to the support body, and an acute angle formed between the guide member and the first direction is variable.

In an embodiment, the first plate may be fixed to the outer surface of the pressing device, and the second plate may be fixed to the inner surface of the guide member.

In an embodiment, the guide member and the flat spring may be each provided in plurality, and the plurality of guide members and the plurality of flat springs may be equally spaced apart from each other around the pressing device.

In an embodiment of the inventive concept, a nasopharyngeal swab sampling apparatus includes: a pressing device; a support device that moves in a first direction relative to the pressing device; and a flat spring that has one end fixed to the pressing device and the other end fixed to the support device, wherein the pressing device includes: a swab coupling member; and a swab which is coupled to the swab coupling member and extends from the swab coupling member in the first direction, wherein the support device includes a guide member that is spaced apart from the swab coupling member in a direction crossing the first direction, and the flat spring coupled to the pressing device and the support device extends from the one end of the flat spring in the first direction and then bent toward the guide member to extend toward the other end of the flat spring in the opposite direction from the first direction.

In an embodiment, the flat spring may include a variable flat spring of which the width is not constant.

In an embodiment, the width of the variable flat spring may increase in a direction from the one end to the other end.

In an embodiment, the variable flat spring may have a trapezoidal shape, and thus the width of the variable flat spring may increase constantly in the direction from the one end to the other end.

In an embodiment, the guide member may have a plate shape, wherein the guide member is inclined to form an acute angle relative to the first direction, and thus the inner surface of the guide member is not parallel to the swab coupling member.

In an embodiment, the nasopharyngeal swab sampling apparatus may further include a driving device which is coupled to the support device and moves the support device.

In an embodiment, the driving device may include: a rotating mechanism configured to rotate the support device around an axis parallel to the first direction; and a position adjusting mechanism configured to move the support device in the first direction.

In an embodiment of the inventive concept, a nasopharyngeal swab sampling method includes: aligning a swab of a robot pressing mechanism in front of the nasal cavity of the human body; moving the swab, which is aligned in front of the nasal cavity, in a first direction to insert the swab into the nasal cavity; and pressing the human body by using the swab in a state in which the swab is inserted into the nasal cavity, wherein the robot pressing mechanism includes: a swab coupling member which extends in the first direction and to which the swab is fixed; a support device that moves in the first direction relative to the swab coupling member; and a flat spring configured to connect the swab coupling member to the support device, wherein the pressing of the human body by using the swab includes: moving the support device in the first direction; and elastically deforming the flat spring by using the support device that moves in the first direction.

In an embodiment, the support device may further include a guide member that is spaced apart from the swab coupling member in a direction crossing the first direction, wherein the flat spring includes: a first plate that has one end fixed to the swab coupling member and extends in the first direction; a second plate that has one end fixed to the guide member and extends in the first direction; and a third plate that has a curved shape and is connected to the other end of the first plate and the other end of the second plate.

In an embodiment, the width of the third plate may be not constant.

In an embodiment, the guide member may be inclined to form an acute angle relative to the first direction, and thus is not parallel to the swab coupling member, and the support device may further include a support body configured to support the guide member, wherein the swab coupling member passes through the support body in the first direction, and the guide member is connected to the support body and rotates relative to the support body.

In an embodiment, the nasopharyngeal swab sampling method may further include rotating the guide member relative to the support body before the swab is inserted into the nasal cavity, thereby changing an angle between the guide member and the support body.

In an embodiment, the nasopharyngeal swab sampling method may further include rotating the swab around an axis parallel to the first direction in a state in which the swab is inserted into the nasal cavity.

Specific features of other embodiments are included in the detailed description and drawings.

### BRIEF DESCRIPTION OF THE FIGURES

The accompanying drawings are included to provide a further understanding of the inventive concept, and are incorporated in and constitute a part of this specification. The drawings illustrate embodiments of the inventive concept and, together with the description, serve to explain principles of the inventive concept. In the drawings:

FIGS. 1 and 2 are perspective views showing a nasopharyngeal swab sampling apparatus according to embodiments of the inventive concept;

FIG. 3 is a perspective view showing a robot pressing mechanism according to embodiments of the inventive concept;

FIG. 4 is an exploded perspective view showing a robot pressing mechanism according to embodiments of the inventive concept;

FIG. 5 is a front view showing a robot pressing mechanism according to embodiments of the inventive concept;

FIG. 6 is a side view showing a robot pressing mechanism according to embodiments of the inventive concept;

FIG. 7 is a plan view showing a flat spring of a robot pressing mechanism according to embodiments of the inventive concept;

FIG. 8 is a plan view showing a portion of a flat spring of a robot pressing mechanism according to embodiments of the inventive concept;

FIG. 9 is a flowchart showing a nasopharyngeal swab sampling method according to embodiments of the inventive concept;

FIG. 10 is a side view showing the nasopharyngeal swab sampling method according to the flowchart of the FIG. 9;

FIG. 11 is a perspective view showing a robot pressing mechanism according to embodiments of the inventive concept;

FIG. 12 is an exploded perspective view showing a robot pressing mechanism according to embodiments of the inventive concept;

FIG. 13 is a side view showing a robot pressing mechanism according to embodiments of the inventive concept;

FIG. 14 is a flowchart showing a nasopharyngeal swab sampling method according to embodiments of the inventive concept; and

FIG. 15 is a perspective view showing the nasopharyngeal swab sampling method according to the flowchart of the FIG. 14.

### DETAILED DESCRIPTION

Preferred examples of the inventive concept will be described with reference to the accompanying drawings so as to sufficiently understand configurations and effects of the inventive concept. However, the inventive concept may not be limited to the embodiments set forth herein but embodied in different forms and diversely modified. Rather, these embodiments are provided so that the disclosure of the inventive concept will be thorough and complete, and will fully convey the scope of the disclosure to a person skilled in the art to which the present disclosure pertains.

Like reference numerals refer to like elements throughout. The embodiments herein will be described with reference to a block diagram, a perspective view, and/or a cross-sectional view as ideal exemplary views of the inventive concept. In the drawing, the thicknesses of regions are exaggerated for effective description of the technical contents. Therefore, regions exemplified in the drawings have general properties, and shapes of the regions exemplified in the drawings are used to illustrate a specific shape of a device region, but not intended to limit the scope of the disclosure. Although various terms are used to describe various components in various embodiments herein, the components should not be limited to these terms. These terms are only used to distinguish one component from another component. The embodiments described and exemplified herein include complementary embodiments thereof.

The terms used herein are used only for explaining embodiments while not limiting the present disclosure. In this specification, the singular forms include the plural forms as well, unless the context clearly indicates otherwise. The meaning of ‘comprises’ and/or ‘comprising’ used herein does not exclude the presence or addition of one or more other components besides the mentioned components.

Hereinafter, the present disclosure will be described in detail by describing preferred embodiments of the inventive concept with reference to the accompanying drawings.

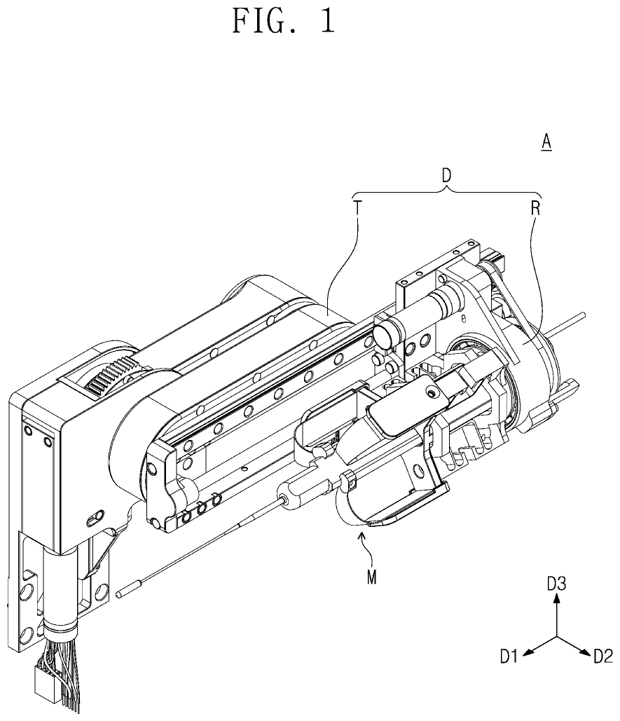

FIGS. 1 and 2 are perspective views showing a nasopharyngeal swab sampling apparatus according to embodiments of the inventive concept.

Hereinafter, D1 may be referred to as a first direction, D2 crossing the first direction D1 may be referred to as a second direction, and D3 crossing both the first direction D1 and the second direction D2 may be referred to as a third direction.

Referring to FIGS. 1 and 2, a nasopharyngeal swab sampling apparatus A may be provided. The nasopharyngeal swab sampling apparatus A may be an apparatus for extracting a sample from the human body so as to test for viruses or the like. More specifically, the nasopharyngeal swab sampling apparatus A may extract a sample from the mucous membrane in the nasal cavity of the human body. The nasopharyngeal swab sampling apparatus A may include a robot pressing mechanism M and a driving device D.

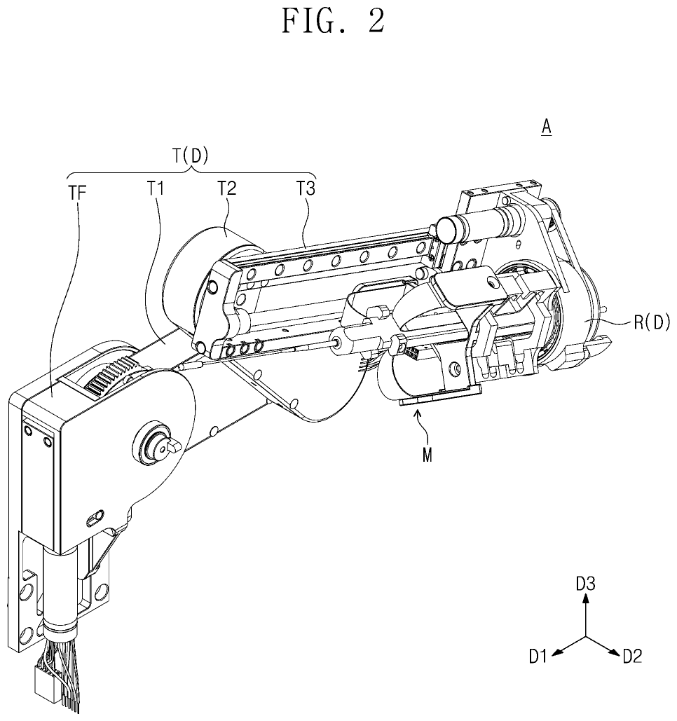

The robot pressing mechanism M may press the target. More specifically, the robot pressing mechanism M may press the target at uniform force. A portion of the robot pressing mechanism M may be inserted into the nasal cavity of the human body. In a state in which a portion of the robot pressing mechanism M is inserted into the nasal cavity of the human body, a sample may be extracted from the mucous membrane in the nasal cavity of the human body. The robot pressing mechanism M may move in various manners. For example, the robot pressing mechanism M may be moved in various directions by the driving device D. The robot pressing mechanism M will be described in detail with reference to FIGS. 3 to 8.

The robot pressing mechanism M has been described as being included in the nasopharyngeal swab sampling apparatus A, but the embodiment of the inventive concept is not limited thereto. That is, the robot pressing mechanism M may be used for other purposes in addition to extracting a sample from the mucous membrane in the nasal cavity of the human body. For example, the robot pressing mechanism M may be utilized in a robot for medical procedures and/or surgeries on the human body. Also, the robot pressing mechanism M may be utilized in semiconductor element-manufacturing equipment. More specifically, the robot pressing mechanism M may be utilized in a robot that presses a semiconductor element to test the semiconductor element. The robot pressing mechanism M may be coupled to another robot for use in other applications. That is, the robot pressing mechanism M may be utilized in various facilities in which targets are pressed by using automated robots. However, hereinafter, the robot pressing mechanism M will be described as being used to extract a sample from the mucous membrane in the nasal cavity of the human body, as a part of the nasopharyngeal swab sampling apparatus A.

The driving device D may move the robot pressing mechanism M. For example, the driving device D may move the robot pressing mechanism M in the first direction D1 or rotate the robot pressing mechanism M around an axis parallel to the first direction D1. To this end, the driving device D may include a rotating mechanism R and a position adjusting mechanism T.

The rotating mechanism R may rotate the robot pressing mechanism M around an axis parallel to the first direction D1. To this end, the rotating mechanism R may be coupled to one side of the position adjusting mechanism T. For example, the rotating mechanism R may be coupled to the rear end of the robot pressing mechanism M. More specifically, the rotating mechanism R may be coupled to a connection member 55 (see FIG. 3) and/or a swab coupling member 11 (see FIG. 3) of the robot pressing mechanism M. Also, the rotating mechanism R may include various structures for rotation. For example, the rotating mechanism R may include an actuator such as a motor and/or a power transmission device such as a gear and a belt. The rotating mechanism R will be described later in detail.

The position adjusting mechanism T may adjust the position of the robot pressing mechanism M. For example, the position adjusting mechanism T may move the robot pressing mechanism M in a direction parallel to the first direction D1 or adjust the height of the robot pressing mechanism M. To this end, the position adjusting mechanism T may include a drive stationary device TF, a first arm T1, a second arm T2, and a third arm T3.

The drive stationary device TF may be fixed at a certain position. The drive stationary device TF may support movements of the first arm T1, the second arm T2, and the third arm T3. The drive stationary device TF may rotate the first arm T1.

The first arm T1 may be rotatably coupled to the drive stationary device TF. The first arm T1 may be rotated around an axis parallel to the second direction D2 by the drive stationary device TF.

The second arm T2 may be rotatably coupled to the first arm T1. The second arm T2 may be rotated around an axis parallel to the second direction D2 by the first arm T1.

The third arm T3 may be rotatably coupled to the second arm T2. The third arm T3 may move the robot pressing mechanism M in the first direction D1 and the opposite direction from the first direction D1. For example, the third arm T3 may be slidingly coupled to the rotating mechanism R and slidingly move the rotating mechanism R in the first direction D1. That is, the rotating mechanism R may be coupled to the side surface of the third arm T3 so as to move in the first direction D1. Accordingly, the robot pressing mechanism M coupled to the rotating mechanism R may move in the first direction D1.

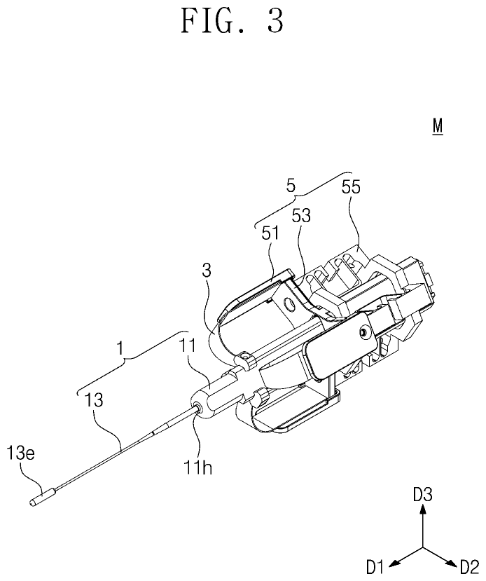

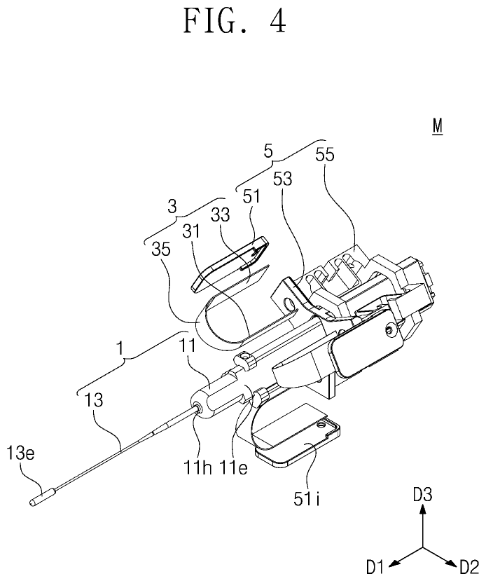

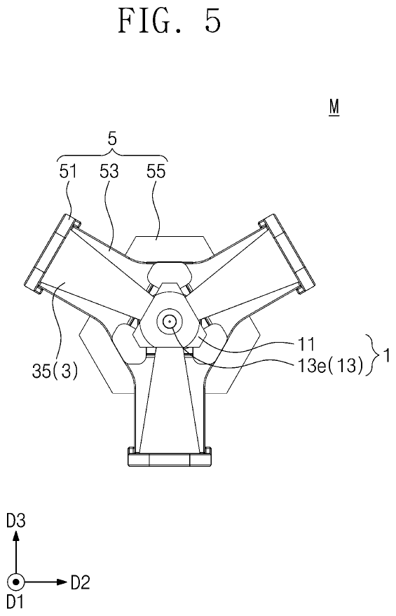

FIG. 3 is a perspective view showing a robot pressing mechanism according to embodiments of the inventive concept, FIG. 4 is an exploded perspective view showing a robot pressing mechanism according to embodiments of the inventive concept, FIG. 5 is a front view showing a robot pressing mechanism according to embodiments of the inventive concept, and FIG. 6 is a side view showing a robot pressing mechanism according to embodiments of the inventive concept.

Referring to FIGS. 3 to 6, the robot pressing mechanism M may include a pressing device 1, a support device 5, and a flat spring 3.

The pressing device 1 may be connected to the support device 5. The pressing device 1 may move in the first direction D1 relative to the support device 5. More specifically, the pressing device 1 is connected to the support device 5 via the flat spring 3, and the pressing device 1 may be moved relative to the support device 5 by elastic deformation of the flat spring 3. The pressing device 1 may be a structure for pressing a target. For example, when the robot pressing mechanism M is utilized in the nasopharyngeal swab sampling apparatus A (see FIG. 1), the pressing device 1 may be a structure that presses the mucous membrane in the nasal cavity of the human body so as to extract a sample. In this case, the pressing device 1 may include a swab coupling member 11 and a swab 13.

The swab coupling member 11 may extend in the first direction D1. The swab coupling member 11 may be connected to the support device 5 via the flat spring 3. In embodiments, as illustrated in FIG. 3, a portion of the swab coupling member 11 may have a triangular prism shape that extends in the first direction D1. Also, another portion of the swab coupling member 11 may have a cylindrical shape that extends in the first direction D1. A portion of the swab coupling member 11 may pass through a portion of the support device 5 in the first direction D1. A swab insertion hole 11h may be provided at one end of the swab coupling member 11. A swab insertion hole 11h may be a hole that is recessed from the one end of the swab coupling member 11 in the opposite direction from the first direction D1.

The swab 13 may extend in the first direction D1. The swab 13 may be coupled to the swab coupling member 11. For example, the swab 13 may be inserted into the swab insertion hole 11h, and coupled and fixed to the swab coupling member 11. The swab 13 may include a sample extractor 13e. The sample extractor 13e may come into contact with the mucous membrane in the nasal cavity of the human body. The sample extractor 13e may press the mucous membrane in the nasal cavity of the human body. This will be described later in detail.

The pressing device 1 has been described as including the swab coupling member 11 and the swab 13, but the embodiment of the inventive concept is not limited thereto. That is, the pressing device 1 may include structures other than the swab 13 depending on the field in which the pressing device 1 is utilized. For example, when the robot pressing mechanism M is utilized in a robot for medical procedures and/or surgeries on the human body, the pressing device 1 may include a knife or the like. Also, when the robot pressing mechanism M is utilized in a robot for a test of a semiconductor device, the pressing device 1 may include a probe tip or the like. When the robot pressing mechanism M is coupled to another robot for use in other applications, the pressing device 1 may include structures suitable therefor. However, hereinafter, the pressing device 1 will be described as including the swab coupling member 11 and the swab 13.

The support device 5 may be connected to the pressing device 1. More specifically, the support device 5 may be connected to the pressing device 1 so as to move in the first direction D1 relative to the pressing device 1. The support device 5 may be connected to the pressing device 1 via the flat spring 3. The support device 5 may be moved relative to the pressing device 1 by elastic deformation of the flat spring 3. The support device 5 may include a guide member 51, a support body 53, and a connection member 55.

The guide member 51 may be spaced apart from the pressing device 1 in a direction crossing the first direction D1. The guide member 51 may be coupled to the flat spring 3. By the flat spring 3, the guide member 51 is coupled to the swab coupling member 11. This will be described later in detail. The guide member 51 may have a plate shape. The plate-shaped guide member 51 may extend in the first direction D1. That is, the guide member 51 may be parallel to the pressing device 1. More specifically, an inner surface 51i of the guide member 51 may be parallel to one side of an outer surface 11e of the swab coupling member 11. For example, when the swab coupling member 11 has a triangular prism shape, the inner surface 51i of the guide member 51 may be parallel to one of the three outer surfaces of the triangular prism shape of the swab coupling member 11. The guide member 51 may be provided in plurality. For example, as illustrated in FIG. 3, three guide members 51 may be provided. The three guide members 51 may be equally spaced apart from each other around the pressing device 1. That is, the three guide members 51 may be spaced about 120 degrees from each other with the pressing device 1 positioned at the center. The three guide members 51 have been illustrated and described, but the embodiment of the inventive concept is not limited thereto. That is, one, two, or four or more guide members 51 may be provided. However, hereinafter, the guide member 51 will be described as a singular form unless there are no other specific circumstances.

The support body 53 may support the guide member 51. For example, the guide member 51 may be coupled to one side of the support body 53. When the guide member 51 is provided in plurality, support bodies 53 may respectively support the plurality of guide members 51. The pressing device 1 may pass through the support body 53 in the first direction D1. To this end, the support body 53 may provide a pressing device through-hole (not designated by a reference numeral). The pressing device through-hole may be greater than the pressing device 1. Accordingly, the pressing device 1 may move relative to the support device 5 in the first direction D1.

The connection member 55 may be coupled to the rear end of the support body 53. The connection member 55 may be coupled to the driving device D (see FIG. 2). For example, the connection member 55 may be coupled to the rotating mechanism R (see FIG. 2). By the connection member 55, the robot pressing mechanism M may be connected to the driving device D.

The flat spring 3 may connect the pressing device 1 to the support device 5. More specifically, the flat spring 3 may connect the pressing device 1 to the support device 5 so that the pressing device 1 moves in the first direction D1 relative to the support device 5. The flat spring 3 may include an elastically deformable material. For example, the flat spring 3 may include metal.

One side of the flat spring 3 may be coupled to the pressing device 1, and the other side of the flat spring 3 may be coupled to the support device 5. For example, one end of the flat spring 3 may be coupled to the swab coupling member 11, and the other end of the flat spring 3 may be coupled to the guide member 51. The flat spring 3 may extend from the one end coupled to the swab coupling member 11 in the first direction D1 and then may be bent toward the guide member 51. The flat spring 3 may be bent to extend toward the other end coupled to the guide member 51 in the opposite direction from the first direction D1. That is, the flat spring 3 may extend from the one end in the first direction D1 and then may be bent to extend toward the other end in the opposite direction from the first direction D1. Here, a portion of the flat spring 3, which includes the one end coupled to the swab coupling member 11, may be referred to as a first plate 31. Also, a portion of the flat spring 3, which includes the other end coupled to the guide member 51, may be referred to as a second plate 33. A portion bent to connect the first plate 31 to the second plate 33 may be referred to as a third plate 35. That is, the flat spring 3 may include the first plate 31, the second plate 33, and the third plate 35.

The first plate 31 may be coupled to the pressing device 1. More specifically, the first plate 31 may be coupled to the outer surface 11e of the swab coupling member 11. The first plate 31 may extend along the outer surface 11e of the swab coupling member 11 in the first direction D1.

The second plate 33 may be coupled to the support device 5. More specifically, the second plate 33 may be coupled to the inner surface 51i of the guide member 51. The second plate 33 may extend along the inner surface 51i of the guide member 51 in a direction in which the guide member 51 extends.

The third plate 35 may connect the first plate 31 to the second plate 33. More specifically, the third plate 35 may be bent to connect one end of the first plate 31 to one end of the second plate 33.

The width of the third plate 35 may not be constant. For example, the width of the third plate 35 may increase in a direction from the first plate 31 to the second plate 33. More specifically, the width of the third plate 35 may increase constantly in the direction from the first plate 31 to the second plate 33. That is, the third plate 35 may have a trapezoidal shape. The third plate 35 may be referred to as a variable flat spring.

The first plate 31, the second plate 33, and the third plate 35 may be integrated with each other. That is, the first plate 31, the second plate 33, and the third plate 35 may be referred to as the respective divided portions of the one flat spring 3.

When the plurality of guide members 51 are provided, the flat spring 3 may also be provided in plurality. That is, the flat springs 3 may be provided as many as the guide members 51. For example, when three guide members 51 are provided, three flat springs 3 may be provided as well. In this case, the three flat springs 3 may be equally spaced apart from each other around the pressing device 1. However, hereinafter, the flat spring 3 will be described as a singular form unless there are no other specific circumstances. The flat spring 3 will be described later in more detail.

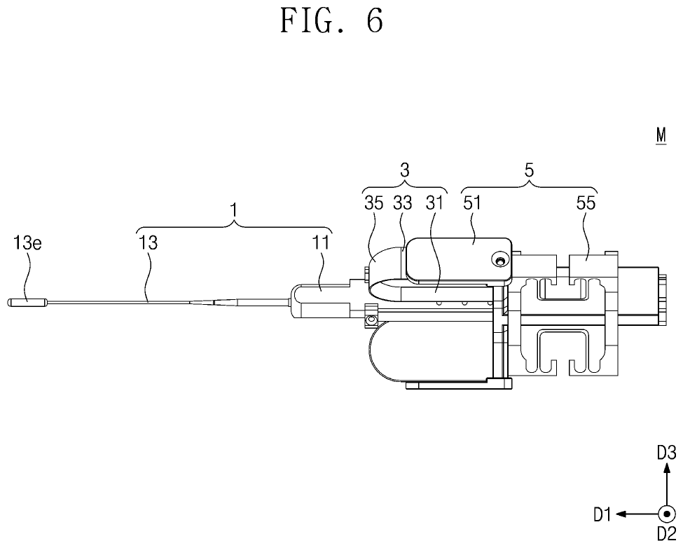

FIG. 7 is a plan view showing a flat spring of a robot pressing mechanism according to embodiments of the inventive concept.

Referring to FIG. 7, as described above, the flat spring 3 may be divided into the first plate 31, the second plate 33, and the third plate 35.

In a region in which the third plate 35 is connected to the first plate 31, the width of the third plate 35 may be substantially identical or similar to that of the first plate 31. Also, in a region in which the third plate 35 is connected to the second plate 33, the width of the third plate 35 may be substantially identical or similar to that of the second plate 33. The width of the second plate 33 may be greater than the width of the first plate 31. Thus, the width of the third plate 35 may not be constant. For example, in a state in which the third plate 35 is unfolded as shown in FIG. 7, the third plate 35 may have a trapezoidal shape.

When the third plate 35 having a variable width is bent within the limit of elasticity as shown in FIG. 6, the first plate 31 moves in the first direction D1, and an elastic restoring force to restore the flat spring 3 to the original shape thereof may be generated. Accordingly, the pressing device 1 coupled to the first plate 31 may move in the first direction D1. When the sample extractor 13e comes into contact with a target such as the mucous membrane in the nasal cavity of the human body, the pressing device 1 may press the target in the first direction D1.

In particular, when the width of the third plate 35 is reduced constantly, the elastic restoring force generated by the flat spring 3 may be constant. More specifically, when the third plate 35 has a trapezoidal shape, in a state in which the third plate 35 is bent about 180 degrees as shown in FIG. 6, the elastic restoring force of the flat spring 3 may be constant irrespective of the deformation positions of the third plate 35. Therefore, the pressing device 1 may press, at a uniform force, the target such as the mucous membrane in the nasal cavity of the human body. Accordingly, it is possible to prevent damage to the mucous membrane in the nasal cavity of the human body.

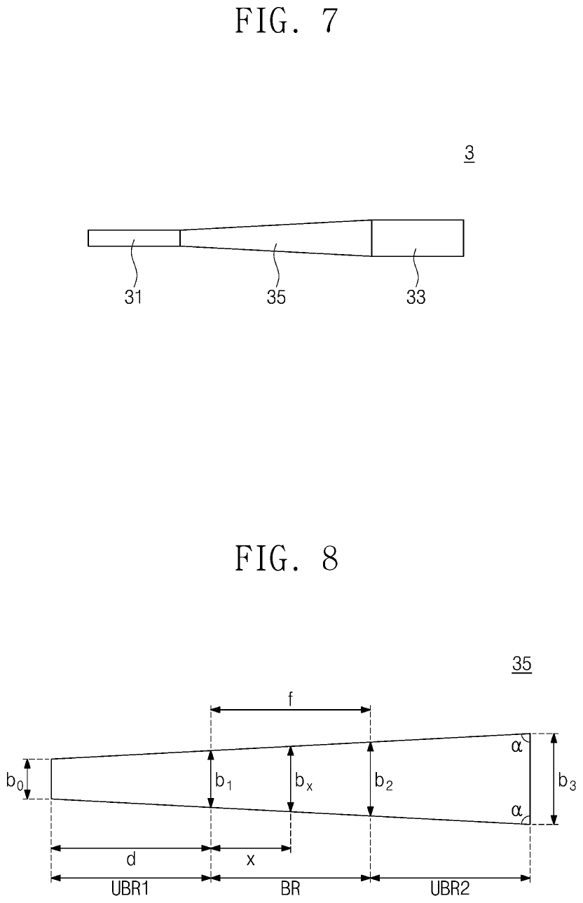

Hereinafter, this will be described with reference to FIG. 8.

FIG. 8 is a plan view showing a portion of a flat spring of a robot pressing mechanism according to embodiments of the inventive concept.

Referring to FIG. 8, the third plate 35 may have a trapezoidal shape. The smallest value of the widths of the third plate 35 may be b₀. The largest value of the widths of the third plate 35 may be b₃.

A bending region BR of the third plate 35 may be bent about 180 degrees as illustrated in FIG. 6. A first non-bending region UBR1 and a second non-bending region UBR2 of the third plate 35 may not be bent.

The smallest value of the widths of the bending region BR may be b₁. The largest value of the widths of the bending region BR may be b₂. The distance from a point, at which the width is b₀, to a start point of the bending region BR may be referred to as d. The width at a point, which is spaced x from the start point of the bending region BR, may be referred to as bₓ. The length of the bending region BR may be referred to as f. Each of the basic angles of the trapezoid may be referred to as α.

Here, bₓ may be calculated as follows.

$$\\ {b}_{x} = {b}_{0} + \frac{2 ( d + x )}{tan α}$$

Also, when the bending region BR of the third plate 35 is bent as illustrated in FIG. 6, the moment may be calculated as follows.

$$\\ M = \frac{EI}{r}$$

In the above equation, r may be the radius of curvature of the third plate 35 that is bent. E may be the modulus of elasticity of the third plate 35. I may be the moment of inertia of the third plate 35. When the cross-section of the third plate 35 has a rectangular shape, the moment of inertia of the third plate 35 may be calculated as follows.

$$\\ I = \text{?} = \text{?}$$

$$\\ \text{?} \text{indicates text missing or illegible when filed}$$

In the above equation, h may represent the height of the third plate 35 on the cross-section.

Thus, when the bending region BR is bent, the internal energy (or the strain energy) of the third plate 35 may be calculated as follows.

$$\\ U = \text{?} = \text{?} = \text{?} + \text{?} + \text{?}$$

$$\\ \text{?} \text{indicates text missing or illegible when filed}$$

That is, when the bending region BR is bent, the internal energy (U) of the third plate 35 may be a linear function with respect to d that is the distance to the start point of the bending region BR.

Also, when the bending region BR of the third plate 35 is bent about 180 degrees as illustrated in FIG. 6, f may be calculated as follows.

f=πr

Therefore, when the bending region BR of the third plate 35 is bent about 180 degrees as illustrated in FIG. 6, the elastic restoring force in the first direction D1 (see FIG. 6) may be calculated as follows.

$$\\ F = \frac{Δ U ( d )}{Δ L} = \frac{Δ U ( d )}{Δ d} \frac{Δ d}{Δ L} = \text{?}$$

$$\\ \text{?} \text{indicates text missing or illegible when filed}$$

Referring to the above equation, when the bending region BR of the third plate 35 is bent about 180 degrees as illustrated in FIG. 6, the elastic restoring force in the first direction D1 may be constant irrespective of the values of d. That is, irrespective of where the bending region BR is located within the third plate 35, the elastic restoring force of the flat spring 3 may be constant. Therefore, irrespective of the amount of deformation of the flat spring 3, the pressing device 1 may press the target at constant force.

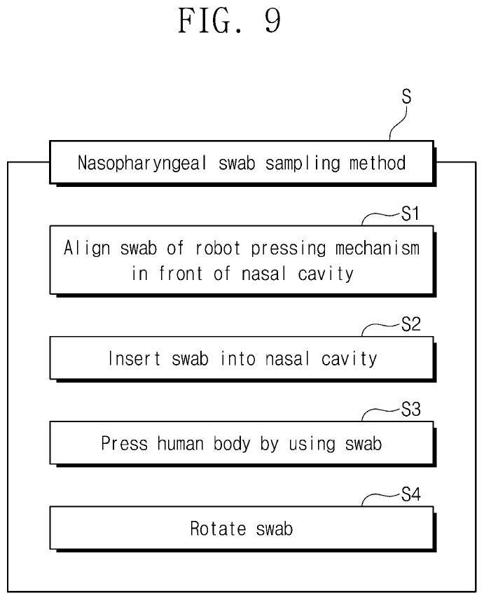

FIG. 9 is a flowchart showing a nasopharyngeal swab sampling method according to embodiments of the inventive concept.

Referring to FIG. 9, a nasopharyngeal swab sampling method (S) may be provided. The nasopharyngeal swab sampling method (S) may be a method for extracting a sample from the mucous membrane in the nasal cavity of the human body by using the nasopharyngeal swab sampling apparatus A (see FIG. 1) described with reference to FIGS. 1 to 9. The nasopharyngeal swab sampling method (S) may include aligning a swab of a robot pressing mechanism in front of the nasal cavity (S1), inserting the swab into the nasal cavity (S2), pressing the human body by using the swab (S3), and rotating the swab (S4).

Hereinafter, the nasopharyngeal swab sampling method (S) of FIG. 9 will be described in detail with reference to FIGS. 1 to 10.

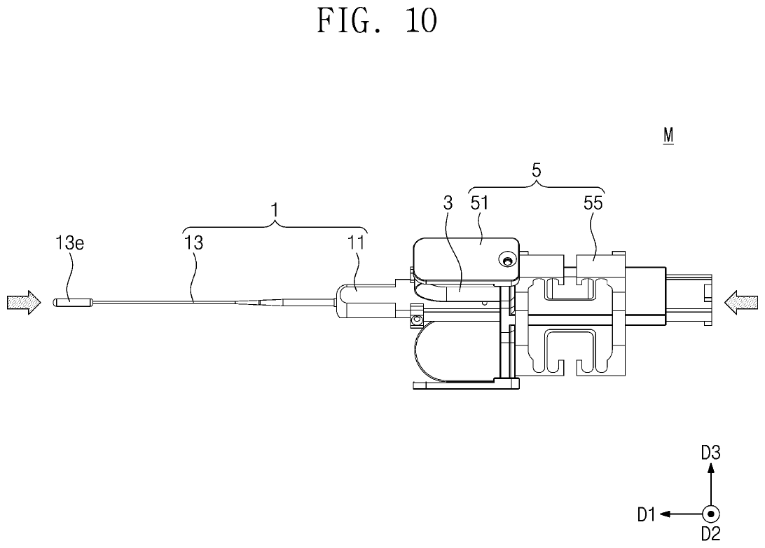

FIG. 10 is a side view showing the nasopharyngeal swab sampling method according to the flowchart of the FIG. 9.

Referring to FIGS. 2 and 9, the aligning (S1) of the swab of the robot pressing mechanism in front of the nasal cavity may include moving the robot pressing mechanism M by using the driving device D so that the swab 13 is aligned in front of the nasal cavity of the human body. More specifically, the robot pressing mechanism M may be moved by using the position adjusting mechanism T, and thus the swab 13 may be aligned in front of the nasal cavity of the human body.

The inserting (S2) of the swab into the nasal cavity may include moving the swab 13 in the first direction D1. More specifically, the robot pressing mechanism M may be moved in the first direction D1 by the third arm T3, and thus the swab 13 may be inserted into the nasal cavity of the human body. The swab 13 may be moved in the first direction D1 until coming into contact with the mucous membrane in the nasal cavity of the human body.

Referring to FIGS. 9 and 10, the pressing (S3) of the human body by using the swab may include moving the support device 5 in the first direction D1 by using the driving device D (see FIG. 2). When the support device 5 moves in the first direction D1, the pressing device 1, which is in contact with the mucous membrane in the nasal cavity of the human body, may press the mucous membrane in the first direction D1. Here, the flat spring 3 may be elastically deformed, and the pressing device 1 may be pushed back in the opposite direction from the first direction D1 relative to the support device 5. When the flat spring 3 is elastically deformed, the elastic restoring force in the first direction D1 may be generated. Therefore, the sample extractor 13e may press the mucous membrane. Here, when the flat spring 3 has a trapezoidal shape, the force applied to the mucous membrane by the sample extractor 13e may be constant. Therefore, it is possible to prevent excessive force from being applied to the mucous membrane, thereby preventing damage to the mucous membrane.

Referring back to FIGS. 2 and 9, the rotating (S4) of the swab may include rotating the swab 13 around an axis parallel to the first direction D1 in a state in which the swab 13 is inserted into the nasal cavity of the human body. More specifically, the rotating mechanism R may rotate the robot pressing mechanism M, and thus the swab 13 may be rotated. According to the rotation of the swab 13, it is possible to smoothly extract a sample from the mucous membrane in the nasal cavity of the human body.

According to the robot pressing mechanism, the nasopharyngeal swab sampling apparatus including the same, and the nasopharyngeal swab sampling method using the same according to embodiments of the inventive concept, the elastic restoring force of the flat spring may be used when a target such as the mucous membrane in the nasal cavity of the human body is pressed by an automated robot. When the elastic restoring force is used, it is possible to press the target while flexibly coping with unexpected movements of the target. Therefore, the robot may be easily controlled. Here, when the flat spring has a trapezoidal shape, it is possible to secure a constant elastic restoring force irrespective of an amount of the deformation of the flat spring. Therefore, the target may be pressed at a constant force. When the robot pressing mechanism is used to extract a sample from the mucous membrane in the nasal cavity of the human body, the mucous membrane may be pressed at a constant pressure, thereby preventing damage to the mucous membrane. That is, during the process of extracting a sample by using the automated robot, it is possible to prevent harm to the human body. Therefore, the reliability of sample extraction by using the automated robot may be enhanced, and the speed of sample extraction may be improved.

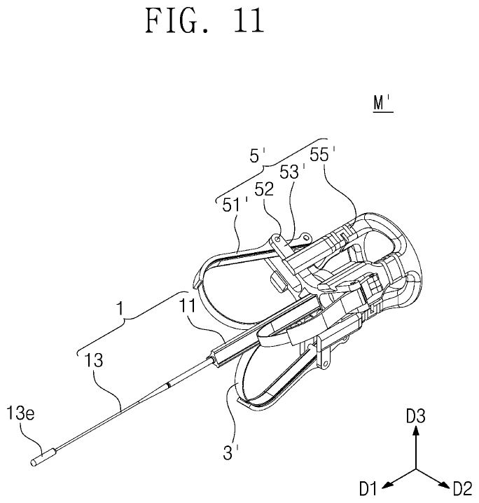

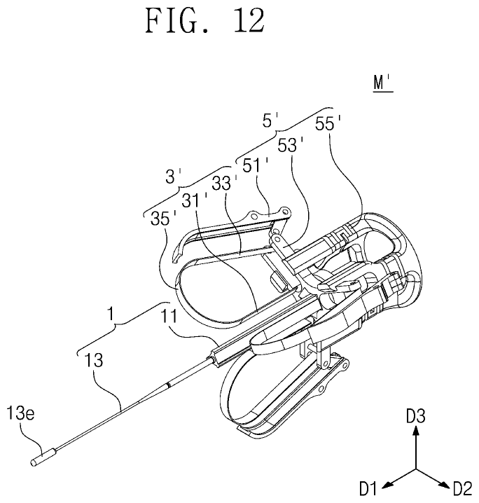

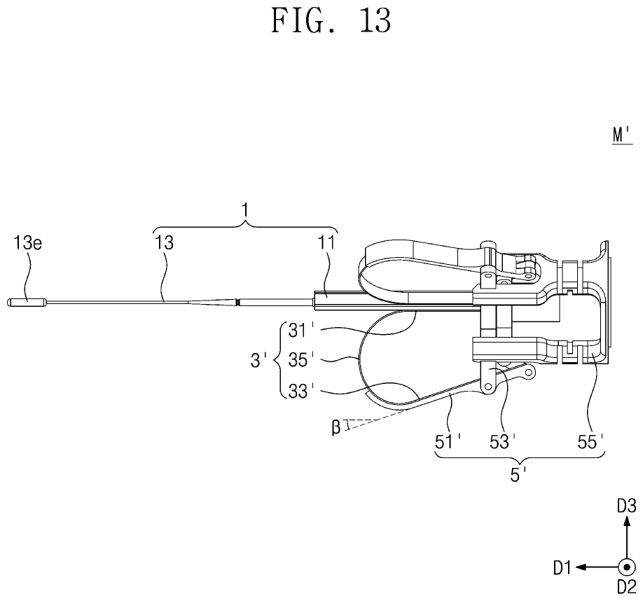

FIG. 11 is a perspective view showing a robot pressing mechanism according to embodiments of the inventive concept, FIG. 12 is an exploded perspective view showing a robot pressing mechanism according to embodiments of the inventive concept, and FIG. 13 is a side view showing a robot pressing mechanism according to embodiments of the inventive concept.

Hereinafter, descriptions substantially identical or similar to those described with reference to FIGS. 1 to 10 will be omitted.

Referring to FIGS. 11 to 13, the robot pressing mechanism M′ may include a pressing device 1, a support device 5′, and a flat spring 3′. The pressing device 1 may be substantially identical or similar to that described with reference to FIG. 3.

The support device 5′ may include a guide member 51′, a support body 53′, and a connection member 55′.

The guide member 51′ may not be parallel to the pressing device 1. For example, the guide member 51′ may form an acute angle R with a first direction D1. More specifically, the inner surface of the guide member 51′ and the outer surface of the pressing device 1 may form the acute angle 3. Therefore, a second plate 33′ coupled to the inner surface of the guide member 51′ may not be parallel to the first direction D1. Also, the first plate 31′ may not be parallel to the second plate 33′. Accordingly, the flat spring 3′ may be bent at an angle other than about 180 degrees.

The support body 53′ may support the guide member 51′. The support body 53′ may rotate the guide member 51′. That is, the guide member 51′ may be rotatably coupled to the support body 53′. For example, the guide member 51′ may be rotatably coupled to the support body 53′ by a rotation hinge 52. Therefore, an angle between the guide member 51′ and the support body 53′ may be changed. Accordingly, the acute angle R between the guide member 51′ and the first direction D1 may be changed. Also, the shape of the flat spring 3′ may also be changed.

The width of the flat spring 3′ may be constant. That is, the flat spring 3′ may have a rectangular shape other than a trapezoidal shape. However, the embodiment of the inventive concept is not limited thereto, and the shape of the flat spring 3′ may be changed according to specific design application.

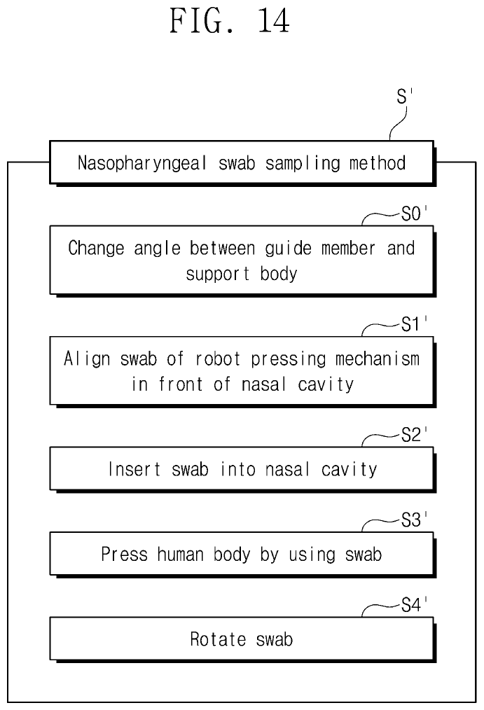

FIG. 14 is a flowchart showing a nasopharyngeal swab sampling method according to embodiments of the inventive concept.

Referring to FIG. 14, a nasopharyngeal swab sampling method (S′) may be provided. The nasopharyngeal swab sampling method (S′) of FIG. 14 may further include changing an angle between a guide member and a support body (S0′), unlike the method described with reference to FIG. 9.

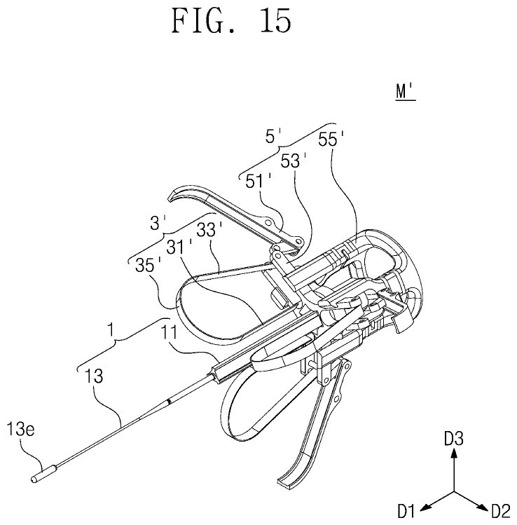

Hereinafter, the nasopharyngeal swab sampling method (S′) of FIG. 14 will be described with reference to FIG. 15.

FIG. 15 is a perspective view showing the nasopharyngeal swab sampling method according to the flowchart of the FIG. 14.

Referring to FIGS. 14 and 15, the changing (S0′) of an angle between the guide member and the support body may be performed before a swab 13 is inserted into the nasal cavity of the human body. The initial shape of the flat spring 3′ may be set as desired by changing the angle between the guide member 51′ and the support body 53′. Accordingly, the elastic restoring force generated by the flat spring 3′ may be controlled.

According to the robot pressing mechanism, the nasopharyngeal swab sampling apparatus including the same, and the nasopharyngeal swab sampling method using the same according to embodiments of the inventive concept, the guide member may be rotated by a desired angle. Therefore, the initial shape of the flat spring may be set as desired. More specifically, in order to adjust the intensity of pressing a target by using the pressing device, the initial shape of the flat spring may be controlled. Through this method, the target may be pressed at an appropriate force. When the robot pressing mechanism is used to extract a sample from the mucous membrane in the nasal cavity of the human body, it is possible to secure an elastic restoring force, which does not damage the mucous membrane, by adjusting the angle of the guide member. Therefore, it is possible to prevent damage to the human body, thereby enhancing the reliability of an automated sample extraction operation.

According to the robot pressing mechanism, the nasopharyngeal swab sampling apparatus including the same, and the nasopharyngeal swab sampling method using the same, it is possible to press a target at uniform force.

According to the robot pressing mechanism, the nasopharyngeal swab sampling apparatus including the same, and the nasopharyngeal swab sampling method using the same, it is possible to prevent damage to the mucous membrane of the human body.

According to the robot pressing mechanism, the nasopharyngeal swab sampling apparatus including the same, and the nasopharyngeal swab sampling method using the same, it is possible to prepare for unexpected movements of the human body.

The effects of the present disclosure are not limited to the aforementioned effects, but other effects not described herein will be clearly understood by those skilled in the art from the following description.

Although the embodiments of the inventive concept are described with reference to the accompanying drawings, those skilled in the art to which the present disclosure pertains will understand that the present disclosure can be carried out in other specific forms without changing the technical idea or essential features. Therefore, the above-described embodiments are to be considered in all aspects as illustrative and not restrictive.

What is claimed is:

1. A robot pressing mechanism comprising:
a pressing device extending in a first direction;
a support device connected to the pressing device and moving in the first direction relative to the pressing device; and
a flat spring configured to connect the pressing device to the support device,
wherein the support device comprises a guide member that is spaced apart from the pressing device in a direction crossing the first direction,
wherein the flat spring comprises:
a first plate coupled to the pressing device and extending in the first direction;
a second plate coupled to the guide member and extending in a direction in which the guide member extends; and
a third plate bent to connect one end of the first plate to one end of the second plate.

2. The robot pressing mechanism of claim 1, wherein the width of the third plate is not constant.

3. The robot pressing mechanism of claim 2, wherein the width of the third plate increases in a direction from the first plate to the second plate.

4. The robot pressing mechanism of claim 3, wherein the third plate has a trapezoidal shape so that the width of the third plate increases constantly in the direction from the first plate to the second plate.

5. The robot pressing mechanism of claim 2, wherein the guide member has a plate shape,
wherein the guide member extends in the first direction, and the inner surface of the guide member is parallel to the pressing device.

6. The robot pressing mechanism of claim 1, wherein the first plate is coupled to the outer surface of the pressing device, and
the second plate is coupled to the inner surface of the guide member,
wherein the inner surface of the guide member is not parallel to the outer surface of the pressing device, and the first plate is not parallel to the second plate.

7. The robot pressing mechanism of claim 6, wherein the support device further comprises a support body configured to support the guide member,
wherein the pressing device passes through the support body in the first direction, and
the guide member is connected to the support body and rotates relative to the support body, and an acute angle formed between the guide member and the first direction is variable.

8. The robot pressing mechanism of claim 1, wherein the first plate is fixed to the outer surface of the pressing device, and
the second plate is fixed to the inner surface of the guide member.

9. The robot pressing mechanism of claim 1, wherein the guide member and the flat spring are each provided in plurality, and
the plurality of guide members and the plurality of flat springs are equally spaced apart from each other around the pressing device.

10. A nasopharyngeal swab sampling apparatus comprising:
a pressing device;
a support device that moves in a first direction relative to the pressing device; and
a flat spring that has one end fixed to the pressing device and the other end fixed to the support device,
wherein the pressing device comprises:
a swab coupling member; and
a swab which is coupled to the swab coupling member and extends from the swab coupling member in the first direction,
wherein the support device comprises a guide member that is spaced apart from the swab coupling member in a direction crossing the first direction, and
the flat spring coupled to the pressing device and the support device extends from the one end of the flat spring in the first direction and then bent toward the guide member to extend toward the other end of the flat spring in the opposite direction from the first direction.

11. The nasopharyngeal swab sampling apparatus of claim 10, wherein the flat spring comprises a variable flat spring of which the width is not constant.

12. The nasopharyngeal swab sampling apparatus of claim 11, wherein the width of the variable flat spring increases in a direction from the one end to the other end.

13. The nasopharyngeal swab sampling apparatus of claim 12, wherein the variable flat spring has a trapezoidal shape, and thus the width of the variable flat spring increases constantly in the direction from the one end to the other end.

14. The nasopharyngeal swab sampling apparatus of claim 10, wherein the guide member has a plate shape,
wherein the guide member is inclined to form an acute angle relative to the first direction, and thus the inner surface of the guide member is not parallel to the swab coupling member.

15. The nasopharyngeal swab sampling apparatus of claim 10, further comprising a driving device which is coupled to the support device and moves the support device.

16. The nasopharyngeal swab sampling apparatus of claim 15, wherein the driving device comprises:
a rotating mechanism configured to rotate the support device around an axis parallel to the first direction; and
a position adjusting mechanism configured to move the support device in the first direction.

17. A nasopharyngeal swab sampling method comprising:
aligning a swab of a robot pressing mechanism in front of the nasal cavity of the human body;
moving the swab, which is aligned in front of the nasal cavity, in a first direction to insert the swab into the nasal cavity; and
pressing the human body by using the swab in a state in which the swab is inserted into the nasal cavity,
wherein the robot pressing mechanism comprises:
a swab coupling member which extends in the first direction and to which the swab is fixed;
a support device that moves in the first direction relative to the swab coupling member; and
a flat spring configured to connect the swab coupling member to the support device,
wherein the pressing of the human body by using the swab comprises:
moving the support device in the first direction; and
elastically deforming the flat spring by using the support device that moves in the first direction.

18. The nasopharyngeal swab sampling method of claim 17, wherein the support device further comprises a guide member that is spaced apart from the swab coupling member in a direction crossing the first direction,
wherein the flat spring comprises:
a first plate that has one end fixed to the swab coupling member and extends in the first direction;
a second plate that has one end fixed to the guide member and extends in the first direction; and
a third plate that has a curved shape and is connected to the other end of the first plate and the other end of the second plate.

19. The nasopharyngeal swab sampling method of claim 18, wherein the width of the third plate is not constant.

20. The nasopharyngeal swab sampling method of claim 18, wherein the guide member is inclined to form an acute angle relative to the first direction, and thus is not parallel to the swab coupling member, and
the support device further comprises a support body configured to support the guide member,
wherein the swab coupling member passes through the support body in the first direction, and
the guide member is connected to the support body and rotates relative to the support body.
